(19) United States
(12) Patent Application Publication
(10) Pub. No.: US 2025/0014721 A1
(43) Pub. Date: Jan. 9, 2025

(54) SYSTEMS, METHODS, AND APPARATUSES FOR IMPLEMENTING A GENERIC UNIFIED DEEP MODEL FOR LEARNING FROM MULTIPLE TASKS

(71) Applicant: Arizona Board of Regents on behalf of Arizona State University, Scottsdale, AZ (US)

(72) Inventors: ZiYu Fan, Tempe, AZ (US); Jianming Liang, Scottsdale, AZ (US)

(73) Assignee: Arizona Board of Regents on behalf of Arizona State University, Scottsdale, AZ (US)

(21) Appl. No.: 18/761,131

(22) Filed: Jul. 1, 2024

Related U.S. Application Data
(60) Provisional application No. 63/524,783, filed on Jul. 3, 2023.

(51) Int. Cl. G16H 30/40; G06T 5/40; G06T 5/92; G06V 10/26; G06V 10/32; G06V 10/764; G06V 10/774; G06V 10/82

(52) U.S. Cl. CPC G16H 30/40; G06T 5/40; G06T 5/92; G06V 10/26; G06V 10/32; G06V 10/764; G06V 10/774; G06V 10/82; G06T 2207/20081; G06T 2207/20084; G06T 2207/30032; G06V 2201/032

(57) ABSTRACT

A generic unified deep model for learning from multiple tasks, in the context of medical image analysis includes means for receiving a training dataset of medical images; training the AI model to generate a trained AI model using a pre-processing operation, a Swin Transformer-based segmentation operation, and a post-processing operation, in which application of a Non-Maximum Suppression (NMS) algorithm generates object detection and classification output parameters for the AI model by removing overlapping detections and selecting a best set of detections according to a determined confidence score for the detections remaining; and outputting the trained AI model for use with medical image analysis.

### CLAIM OF PRIORITY

This application claims the benefit of U.S. Provisional Patent Application No. 63/524,783, filed Jul. 3, 2023, entitled “SYSTEMS, METHODS, AND APPARATUSES FOR IMPLEMENTING A GENERIC UNIFIED DEEP MODEL FOR LEARNING FROM MULTIPLE TASKS”, the disclosure of which is incorporated by reference herein in its entirety.

### GOVERNMENT RIGHTS AND GOVERNMENT AGENCY SUPPORT NOTICE

This invention is made with government support under R01 HL128785 awarded by the National Institutes of Health. The government has certain rights in the invention.

### COPYRIGHT NOTICE

A portion of this patent document contains material which is subject to copyright protection. The copyright owner has no objection to the facsimile reproduction by anyone of the disclosure as it appears in the Patent and Trademark Office patent file or records, but otherwise reserves all copyright rights whatsoever.

### TECHNICAL FIELD

Embodiments of the invention relate generally to the field of medical imaging and analysis using convolutional neural networks and transformers for the classification and annotation of medical images, and more particularly, to systems, methods, and apparatuses for implementing a generic unified deep model for learning from multiple tasks, in the context of medical image analysis.

### BACKGROUND

Machine learning models have various applications to automatically process inputs and produce outputs considering situational factors and learned information to improve output quality. One area where machine learning models, and neural networks in particular, provide high utility is in the field of processing medical images.

Within the context of machine learning and deep learning specifically, a Convolutional Neural Network (CNN, or ConvNet) is a class of deep neural networks, very often applied to analyzing visual imagery. Convolutional Neural Networks are regularized versions of multilayer perceptrons. Multilayer perceptrons are fully connected networks, such that each neuron in one layer is connected to all neurons in the next layer, a characteristic which often leads to a problem of overfitting of the data and the need for model regularization. Convolutional Neural Networks also seek to apply model regularization, but with a distinct approach. Specifically, CNNs take advantage of the hierarchical pattern in data and assemble more complex patterns using smaller and simpler patterns. Consequently, on the scale of connectedness and complexity, CNNs are on the lower extreme.

Unfortunately, prior known techniques regardless of using unsupervised or supervised learning modes fail to yield trained AI models with sufficient reliability to correctly identify the presence of colon polyps, which leads directly to a failure to improve the prevention rate of colon cancer.

What is needed is an improved technique for the generation and training of AI models which are both sufficiently generic such that they can learn from multiple tasks and yet trainable and tunable to produce superior reliability in the identification of colon polyps, which in turn will aid in the prevention of colon cancers.

The present state of the art may therefore benefit from the systems, methods, and apparatuses for implementing a generic unified deep model for learning from multiple tasks, in the context of medical image analysis, as is described herein.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 depicts an exemplary model which requires modification individually, on a per-model and per-task basis, for each task execution, i.e., for training models for three different tasks.

FIG. 2 depicts an exemplary network structure of the disclosed Swin-Unet+ model, which extends functionality beyond Swin Transformer.

FIG. 3A depicts Table 1, as set forth at element 305, showing a comparison of model accuracies for classification tasks.

FIG. 3B depicts a comparison chart of model accuracies for classification tasks, in which the vertical bars represent the average accuracy of the different models.

FIG. 4A depicts Table 2, showing a P-value comparison of SOTA models results and results for the best performance model (Swin-Unet+) in the colonoscopy quality assessment and colonoscopy polyp image classification tasks.

FIG. 4B depicts Table 3, showing a comparison of the disclosed model (Swin-Unet+) with SOTA models on the task of colon polyp segmentation tasks.

FIG. 4C depicts Table 4, as set forth at element 415, showing a comparison of the standard deviation of the model results.

FIG. 4D depicts Table 5, showing a P-value comparison results of SOTA model results with results for the best performing model (Swin-Unet+) on the colon polyp segmentation task.

FIG. 4E depicts a comparison chart of model dice coefficients for segmentation tasks evaluated across different models.

FIG. 5A depicts Table 6, showing a comparison of the disclosed model (Swin-Unet+) with a SOTA model on the task of colon polyp object detection.

FIG. 5B depicts Table 7, showing a comparison of the standard deviation of the model results.

FIG. 5C depicts Table 8, showing a P-value comparison results of the disclosed model (Swin-Unet+) and model nnDetection with the best performing model (YOLOV8) on the colon polyp object detection task.

FIG. 5D depicts a comparison chart of model average precision for detection tasks, as evaluated across different models.

FIG. 6 depicts a visual presentation, in which the first row shows the attentional heat (CAM) map drawn from the network, the second row shows the segmented ground truth, the third row shows the segmentation results predicted by the network, and the fourth row shows the results of intestinal polyp detection by the network.

### DETAILED DESCRIPTION

Described herein are systems, methods, and apparatuses for implementing a generic unified deep model for learning from multiple tasks, in the context of medical image analysis.

### INTRODUCTION

Colonoscopy quality often determines colon cancer prevention. However, existing models are still insufficient for the correct identification of colon polyps, and fail to improve the prevention rate of colon cancer. Described herein is a solution to the critical challenge of early detection and accurate diagnosis of intestinal polyps to prevent colorectal cancer. To achieve this goal, a novel multi-task model Swin-Unet+ is described that combines the strengths of Swin-Transformer and U-Net architectures for polyp classification, segmentation, and detection.

In the description that follows various methods are discussed, including nnUNet, TransUNet, and YoLov8. These techniques are then contrasted with the disclosed model, which leverages the powerful representation capabilities of the Swin-Transformer for high-level feature extraction and the robustness of U-Net for accurate segmentation and localization, according to embodiments of the invention. The novel fusion strategy described in greater detail below enables the joint learning of polyp-specific features across tasks, resulting in improved performance. To validate the efficacy of the disclosed model, extensive experiments were conducted on two benchmark datasets, Kvasir-SEG and BKAI-IGN.

The methodologies set forth herein, implemented via the disclosed model, achieve state-of-the-art performance, outperforming existing methods in terms of precision, recall, and a machine learning evaluation metric that measures a model's accuracy, known as an F1 score. For the colon polyp classification task, the accuracy of the disclosed model can reach 95.2%. For the colon polyp segmentation task, the disclosed model can achieve 91.7% for DICE and 85.3% for Jaccard. For the colon polyp target detection task, the Mean Average Precision of the disclosed model reaches 88.3%, the Recall value reaches 84.7%, and the precision value reaches 86.2%. Specifically, remarkable improvements are demonstrated over each of nnUNet, TransUNet, and YoLov8 on both datasets.

Deep learning offers expert-level and sometimes even super-expert-level performance, yet, achieving such performance demands a massively annotated dataset for training.

In the context of medical imaging, there are a variety of modalities and many applications, yielding numerous (different) datasets. Unfortunately, these datasets are individually small, inconsistent in disease coverage, and heterogeneous in expert annotations.

Therefore, described herein is a novel method which is specially configured to utilize many (different) image datasets and their associated heterogeneous annotations for classification, localization, and segmentation across imaging modalities to pre-train generic source models that are more robust, generalizable, and transferable to application-specific target tasks. Stated differently, the methodologies set forth herein provide a novel method that pre-trains deep models by utilizing all accessible annotations for classification, localization, and segmentation across imaging modalities, resulting in pre-trained models that are more generalizable and transferrable to a variety of image analysis tasks, while offering superior and robust performance.

From a high level, the disclosed techniques include at least the following improvements: (1) Integrate Functions, providing: Image-level classification, Organ/Lesion localization, Lesion-level classification, Organ/Lesion segmentation; (2) Unify Modalities, including: X-rays, CT (computed tomography), MRI, Colonoscopy, etc.; and (3) Aggregate Annotations, including: Image-level label, Organ/Lesion bounding box, Organ/lesion markers, Organ/Lesion masks.

Benefits from Modality Unification, Function Integration, and Annotation Aggregation include at least: enlarged data size, diversified patient populations, accrued knowledge from more experts, trained generic source models which are strong in generalizability, transferability, and robustness, yield application-specific target models which are superior in task performance and robust in imbalanced datasets (biases).

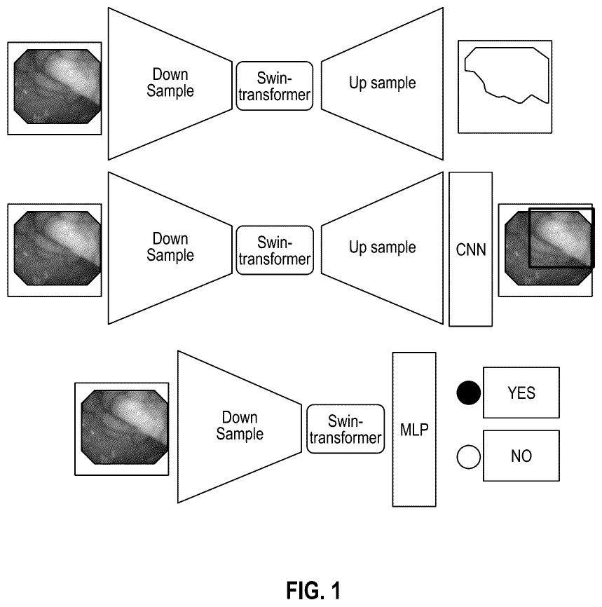

FIG. 1 depicts an exemplary model which requires modification individually, on a per-model and per-task basis, for each task execution, that is, FIG. 1 depicts the preparation, i.e., training models for three different tasks. The models are modified separately for each model and each task, for each task they perform. Consequently, information is not shared between tasks. From top to bottom, depicted are commonly used Swin-Transformer-based segmentation networks, object detection networks, and classification networks. Among them, for the object detection network, adding a layer of CNN to the last layer in the Darknet network often presents excellent performance. For the classification task, adding a multi-layer classifier (MLP) at the last layer shows excellent performance in the binary classification of colon polyps.

These three models all exhibit excellent performance, raising the question “Is it possible to combine these three models of different tasks in the same network structure?” In response to this question, the disclosed model is specially configured to implement an end-to-end model within which the auxiliary information sharing that exists between tasks jointly improves each task's accuracy and each task can provide useful information to each other, an idea that is common in multi-task learning, i.e., multiple tasks jointly constrain the gradient direction.

The incidence of colorectal cancer (CRC) ranks third in the world for many years in terms of cancer incidence. Therefore, how to prevent colorectal cancer has become a public health issue worldwide. Studies have pointed out that 95% of colorectal cancers are caused by colorectal polyps. Timely detection and removal of colorectal polyps can greatly reduce the incidence of colorectal cancer. Currently, the most effective way to prevent colorectal cancer is a regular colonoscopy check to facilitate detection followed by performance of a polypectomy in time to facilitate cancer prevention. With the advent and popularity of painless colonoscopies, the acceptance of this test has increased. However, in the past, the detection of polyps was judged by manual observation by endoscopists, which largely relied on the experience and ability of doctors and required significant time and energy. Exacerbating this issue is the reality that visual fatigue during work leads to misdiagnosis or missed diagnosis. The computer-aided detection system can display the location of polyps in the colonoscopy video in real-time, assisting the endoscopist in making judgments, thus reducing the probability of missed or misdiagnosed polyps.

In recent years, the application of artificial intelligence (AI) and machine learning (ML) techniques, particularly deep learning, has revolutionized the field of medical imaging. Convolutional Neural Networks (CNNs) have demonstrated remarkable performance in various medical image analysis tasks, such as polyp detection in colonoscopy videos, and assessment of image informativeness in colonoscopy. CNNs have been developed and fine-tuned for specific medical imaging applications, resulting in improved diagnostic capabilities and better patient outcomes. And yet, despite the success of CNNs, they still face challenges in terms of scalability, adaptability, and generalization to new tasks or imaging modalities.

To address these issues, researchers have investigated different architectures and training strategies, such as self-supervised learning, transfer learning, and multi-task learning. Recently, the introduction of Transformer models, which leverage self-attention mechanisms, have shown promising results in various domains, including natural language processing (NLP) and computer vision. These models have been adapted for medical imaging tasks, yielding impressive results in classification, segmentation, and detection tasks.

The Swin Transformer is a more recent Transformer architecture that has gained attention for its superior performance in various computer vision tasks. It divides an image into non-overlapping patches and hierarchically processes them, making it well-suited for handling large-scale images, such as medical scans. Researchers have combined the Swin Transformer with established medical imaging architectures, such as the U-Net architecture, to create robust and versatile models capable of handling multiple tasks, including classification, segmentation, and detection.

The multi-task learning approach is particularly attractive in the medical imaging domain, as it allows for the joint learning of related tasks, potentially leading to better performance and generalization. Moreover, this approach can help to overcome the limited availability of annotated medical imaging data, as the model can leverage shared features and representations learned across different tasks.

One of the critical factors that contribute to the performance of deep learning models in medical imaging is the choice of training strategy. Researchers have explored different pre-training methods, such as discriminative, restorative, and adversarial learning, to boost the performance of models and facilitate transfer learning. These strategies can be employed in the context of the Swin Transformer and U-Net-based multi-task models, potentially leading to improved performance and adaptability across different tasks and imaging modalities.

In summary, the integration of the Swin Transformer and U-Net architectures offers a promising direction for developing multi-task models for medical image analysis. By leveraging recent advancements in self-supervised learning, transfer learning, and multi-task learning, these models can overcome the limitations of traditional CNNs, ultimately contributing to improved diagnostic capabilities and patient outcomes.

With reference again to FIG. 1, an overview of the recent progress of the Transformer model for medical imaging is provided, with special attention paid to the application of the Swin Transformer and U-Net in classification, segmentation, and detection tasks, each of which are discussed in conjunction with a combined Swin-Unet-based implementation of a multi-tasking model. An overview of self-supervised learning, transfer learning, and multi-task learning is further presented to provide a comprehensive understanding of the state-of-the-art in this field.

Next, the performance of these prior known models is reviewed in the context of various target medical imaging tasks, including polyp detection, pulmonary embolism detection, and patch order prediction, and appearance recovery. While polyp detection is used as an example in the abstract, the review, evaluations, and results cover a broad range of medical imaging tasks to showcase the versatility and potential of these models in clinical settings.

Overall, the review highlights the significant progress made in applying Transformer models to medical imaging tasks and their potential impact on improving patient outcomes.

The exemplary architecture depicted by FIG. 1 demonstrates the research motivation. For instance, it is known that a Swin Transformer shows excellent performance in classification, segmentation, and object detection tasks. However, there is no Swin transformer-based network structure that can unify the three tasks in the current research field. To solve this problem, disclosed herein is a novel implementation for a Swin-Unet-based multi-task learning model, which is called the “Swin-Unet+” model in the description that follows.

### Neural Networks:

Medical image segmentation is a critical task in medical image analysis that aims to extract the regions of interest from medical images. Over the years, researchers have proposed several techniques to tackle this problem. Described below is a high-level overview of the recent advancements in medical image segmentation.

Convolutional Neural Networks (CNNs): CNNs have been widely used in medical image segmentation due to their ability to extract features from images effectively. Many CNN-based models have been proposed, including U-Net, SegNet, and FCN. These models have achieved state-of-the-art performance in several medical image segmentation benchmarks.

Deep Attention Networks: Deep attention networks, such as Attention U-Net, have been proposed to improve the accuracy of medical image segmentation. These models use attention mechanisms to focus on the relevant features and suppress irrelevant ones, which helps to improve segmentation accuracy.

Graph Neural Networks (GNNs): GNNs have shown great potential in medical image segmentation, especially in segmenting 3D medical images. Various techniques have sought to utilize a GNN-based model for 3D medical image segmentation that achieved state-of-the-art performance on several benchmarks.

Generative Adversarial Networks (GANs): GANs have been used in medical image segmentation to generate high-quality segmentation masks. Various techniques have sought to utilize a GAN-based model for brain tumor segmentation that achieved state-of-the-art performance on several benchmarks.

Transformer-based Models: Recently, transformer-based models, such as Swin Transformer, have been proposed for medical image segmentation. These models use a hierarchical structure to process the image patches, which enables the model to capture spatial information effectively and achieve state-of-the-art performance in many benchmarks. Swin Transformer is a recently proposed transformer-based model that has shown great potential in various computer vision tasks.

An overview of recent advancements in Swin Transformers is provided, as follows:

Swin Transformer: Swin Transformer is a hierarchical transformer-based model that uses shifted windows to process the image patches. Swin Transformer employs a multi-scale architecture to capture spatial information effectively and achieves state-of-the-art performance in several computer vision benchmarks.

Swin Transformer in Object Detection: Swin Transformer has achieved state-of-the-art performance in several object detection benchmarks. Various techniques have sought to utilize a Swin Transformer-based object detection model that achieved better results than the state-of-the-art models on several datasets.

Swin Transformer in Image Segmentation: Swin Transformer has achieved state-of-the-art performance in several image segmentation benchmarks. Various techniques have sought to utilize a Swin Transformer-based model for semantic segmentation that achieved better results than the state-of-the-art models on several datasets.

Swin Transformer in Medical Image Segmentation: Swin Transformer has also shown great potential in medical image segmentation. Various techniques have sought to utilize a Swin U-Net for medical image segmentation that achieved better results than the state-of-the-art models on several datasets.

Swin Transformer in Image Restoration: Swin Transformer has been used in image restoration tasks, such as super-resolution and denoising. Various techniques have sought to utilize a SwinIR for image restoration that achieved state-of-the-art performance on several benchmarks.

In the past few years, Multi-Task Learning (MTL) has made remarkable progress in medical image analysis. Some state-of-the-art multi-tasking models have been proposed and applied to different tasks, such as image classification, segmentation, and detection. However, Swin-Unet, a Transformer-based multitasking framework model, has shown significantly superior performance in intestinal polyp classification, intestinal polyp segmentation, and intestinal polyp detection.

Based on the structure of Swin-Unet, a network structure is specially designed and implemented that can be used for multiple tasks, referred to herein as the Swin-Unet+ model. Compared with the current state-of-the-art multitasking models, Swin-Unet+has several advantages, including:

Efficient feature extraction: Swin-Unet+leverages the hierarchical feature extraction capabilities of the Swin Transformer to capture local and global information more efficiently. Compared to traditional convolutional neural networks (CNNs), Swin Transformer provides a higher perceptual field and stronger feature representation, thus improving overall performance.

End-to-end multi-task learning: Swin-Unet+integrates multiple tasks (e.g., classification, segmentation, and detection) into a unified network, enabling end-to-end learning. This reduces the number of parameters, reduces the risk of overfitting, and increases the speed of training and inference.

Adaptive task weights: Swin-Unet+uses an adaptive task weight assignment strategy that automatically adjusts the loss weights according to the difficulty and importance of each task. This allows the model to balance performance across tasks and avoid the performance of one task being affected by others.

Good generalization capability: Swin-Unet+has been extensively validated via experimentation on several medical image datasets and has achieved excellent results in various metrics. The results show that the model has a strong generalization ability and can effectively cope with the diversity challenges in practical applications.

In summary, the Swin-Unet+ model disclosed herein, as a multi-task framework model that integrates intestinal polyp classification, intestinal polyp segmentation, and intestinal polyp detection, shows superior performance in comparison with current state-of-the-art multi-task models. This provides strong support for future research and applications in the field of medical image analysis.

### Method:

Described below is the novel implementation and methodology for colon polyp segmentation and detection using a Swin Transformer which consists of three sub-operations, including: (1) preprocessing, (2) Swin Transformer-based segmentation, and (3) post-processing. The detailed methodology is provided below, as follows.

Preprocessing: The input colonoscopy images are preprocessed to remove the artifacts and enhance the contrast. The preprocessing operation includes image resizing, normalization, and contrast enhancement. The “Contrast Limited Adaptive Histogram Equalization” or “CLAHE” method is utilized for contrast enhancement.

Swin Transformer-based Segmentation: A Swin Transformer-based segmentation network is utilized for colon polyp segmentation. The Swin Transformer-based network consists of a backbone network and a segmentation head. The backbone network is a hierarchical network that uses shifted windows to process the image patches. The segmentation head is used to generate the segmentation masks.

Post-processing: The post-processing operation includes noise reduction, object detection, and classification. Application of the Non-Maximum Suppression (NMS) algorithm is utilized for object detection and classification. The NMS algorithm removes the overlapping detections and selects the best detections based on the confidence score.

This method uses the U-Net architecture with up-sampling and down-sampling, but the difference is that the middle bottleneck layer of U-Net is replaced with the Swin-transformer structure, which is specially configured to better capture the contextual structure relationship to extract more global features from the dataset as a whole.

The number of Swin transformers decreases and the perceptual range of each patch expands (the number of patches remains the same) as the depth of the network increases. Unlike ResNet which relies mainly on convolutional kernels, Swin relies on transform, which is designed to facilitate the hierarchy of Swin Transformers construction and to be able to adapt to multiple scales of visual tasks.

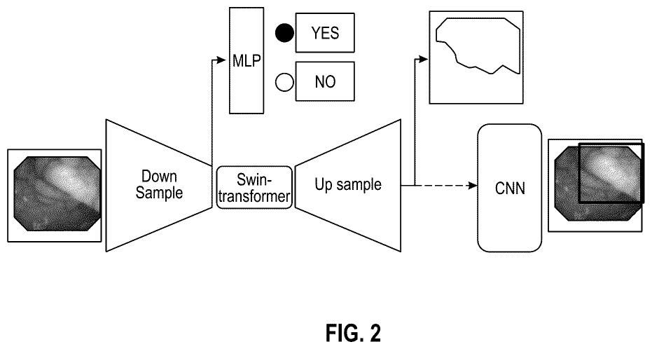

FIG. 2 depicts an exemplary network structure of the disclosed Swin-Unet+model, according to embodiments of the invention, which extends functionality beyond prior known Swin Transformer frameworks and implementations.

The entire network structure shares the encoder. For the classification part, an MLP is connected from the encoder of U-Net for classification, while the segmentation is performed at the pixel level classification head, and the CNN is connected at the end for detection of bounding box detection points for output.

The segmentation task is done directly by down-sampling/up-sampling the output feature map to compare with the ground truth and using the Dice and CE loss function as the optimization function.

The object detection uses this structure followed by multilayer convolution as the output. In one embodiment, the convolution is 7*7*6 (where 7 represents the division of the graph into 7*7 blocks of regions, 6 represents the (x,y,w,h,c) of the bounding box (5) and the class of polyps (1)).

Model Integration: According to certain embodiments, to make the model more consistent overall, the rest of the model remains un-transformed, by replacing only the final output layer. The backbone network of the structure, used to do feature extraction network and representing a part of the network, is generally used for front-end extraction of picture information and generating feature map. The backbone network picks up the task-specific network, providing additional fine-tuning, to the extent feasible for a target-task. The specific network structure connected according to the task is visualized in FIG. 2, in which the end of each task gives a performance boost to the backbone and a positive incentive to subsequent downstream tasks.

In the disclosed multi-task model, a novel fusion strategy is utilized to jointly learn polyp-specific features for classification, segmentation, and detection tasks. The fusion strategy is inspired by the concept of Transferable Visual Words (TVW). The TVW approach enables the model to learn high-level semantic information from anatomical patterns, which can be transferred across tasks.

The fusion process of the disclosed model consists of the following key operations:

Feature Extraction: The Swin-Transformer is utilized as the backbone for extracting high-level features from input images. The Swin-Transformer captures the global context and fine-grained details, which are essential for polyp representation.

Task-specific Branches: To address the three tasks, separate branches are introduced after the Swin-Transformer. For classification, a global average pooling layer followed by a fully connected layer is employed. For segmentation and detection, UNet-like branches are added to capture local context and generate accurate polyp masks and bounding boxes, respectively.

Transferable Visual Words: To facilitate the sharing of semantic information across tasks, a TVW module is integrated according to certain embodiments. This module is placed between the Swin-Transformer and task-specific branches, allowing the model to learn a shared set of visual words that can be transferred across tasks. The TVW module includes a dictionary learning layer and a sparse coding layer, which jointly enable the model to discover and exploit the most discriminative anatomical patterns for each task.

Joint Optimization: To train the model end-to-end, a multi-task loss function is utilized which is specially configured to combine the individual losses for classification, segmentation, and detection. This joint optimization encourages the model to learn shared features while minimizing the task-specific losses.

By fusing the tasks in this manner, the disclosed model effectively learns and transfers semantic information across the tasks, leading to improved performance in polyp classification, segmentation, and detection. The fusion strategy enables the disclosed model to outperform previously known state-of-the-art methods, demonstrating its potential for practical application in clinical settings.

### Experiment:

Method overview: For the sake of experimental evaluation, the disclosed Swin-Unet+ model is applied to three different tasks: classification, segmentation, and detection of intestinal polyps. This section provides an overview of the datasets used and the experimental setup for each task. The detailed method for each task will be described in the following sections.

### Dataset:

Classification task: A publicly available dataset was selected for intestinal polyp classification, which included images of intestinal polyps and normal intestinal tissues. To evaluate the performance of the model on mass classification and classification with or without intestinal polyps, the dataset was processed by grouping the intestinal polyp images by mass, while including the normal tissue images as another group.

Segmentation task: The Kvasir-SEG and BKAI-IGH NeoPolyp datasets were utilized, each of which contained medical images of intestinal polyps with segmentation labels.

Detection task: The KUMC dataset was utilized, which provides medical images of intestinal polyps with multiple pathological types and the corresponding detection labels.

### Experimental Setup:

The disclosed Swin-Unet+ model was applied to the classification, segmentation, and detection tasks, respectively, and compared them with other mainstream methods. For classification tasks, accuracy was utilized as the evaluation measure. For segmentation tasks, the DICE coefficient and Jaccard value were utilized as metrics. For the target detection task, mean Average Precision was utilized as a measure. For the experiments, a five-fold cross-validation was utilized to divide the dataset into a training set and a test set. The training process used the Adam optimizer with a learning rate of 1e-4 and a batch size of 16, with all experiments being conducted on an NVIDIA Geforce RTX 3090 GPU.

### Results:

General overview of the results Classification task: The disclosed Swin-Unet+ model achieved 93.2% accuracy in mass classification and the Swin-Unet+ model achieved 96.5% accuracy in the classification of the presence or absence of intestinal polyps. Compared with other methods, the disclosed Swin-Unet+ model showed a significant performance improvement on the classification task.

Segmentation task: The Dice coefficient of Swin-Unet+was 0.87 and Jaccard coefficient was 0.78 on the Kvasir-SEG dataset, and the Dice coefficient was 0.89 and Jaccard coefficient was 0.80 on the BKAI-IGH NeoPolyp dataset. Swin-Unet+ showed higher segmentation performance on both data sets compared with other methods.

Detection task: On the KUMC dataset, the average precision (mAP) of Swin-Unet+reached 0.92, which is a significant improvement in detection performance compared with other methods.

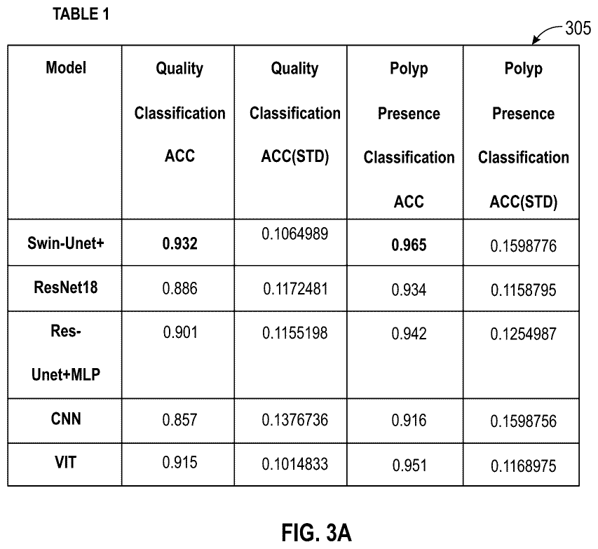

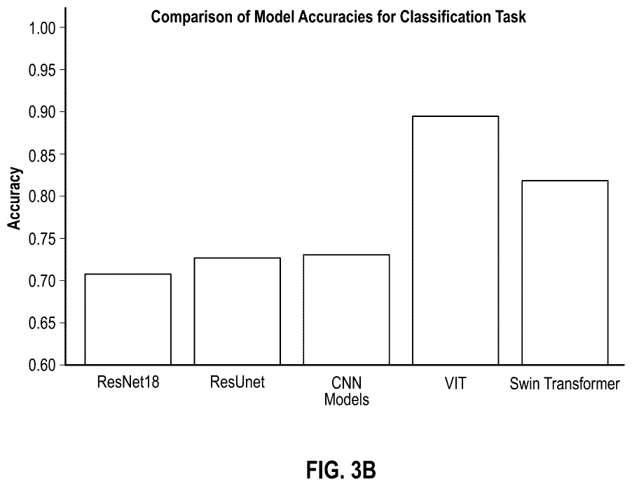

FIG. 3A depicts Table 1, as set forth at element 305, showing a comparison of model accuracies for classification tasks.

Classification Task: The disclosed Swin-Unet+ model was compared with each of ResNet18, ResNetUnet+MLP, CNN, and Vision Transformer for the classification task. Table 1 provides a comparison illustrating the performance of each model:

The comparison at Table 1 of colonoscopy quality assessment and colonoscopy polyp image classification provides the following results: The first and second columns are the results of the colonoscopy image quality assessment classification task. The third and fourth columns are the results of the colon polyp presence classification task. The first column shows the average accuracy (ACC) results of the Swin-Unet+ and SOTA models for the colonoscopy image quality assessment classification task. The third column shows the average accuracy (ACC) of the Swin-Unet+ and SOTA models for the polyp presence in the Polyp Presence Classification task. The second and fourth columns are the standard deviation (STD) of the model results, which are used to show the degree of dispersion of the results, thereby illustrating the reliability and stability of the results.

FIG. 3B depicts a comparison bar chart of model accuracies for classification tasks, in which the vertical bars represent the average accuracy of the different models. The Swin Transformer represents the disclosed model (Swin-Unet+).

From the comparison at Table 1, it is evident that the Swin-Unet+outperforms other models in both quality classification and polyp presence classification tasks. The superiority of the disclosed Swin-Unet+ model is attributed to the combination of the Swin Transformer and U-Net architecture, which allows the disclosed Swin-Unet+ model to capture both local and global features effectively. The Swin Transformer provides excellent feature extraction capabilities, while the U-Net structure of the disclosed Swin-Unet+ model allows the disclosed model to maintain spatial information. This combination results in a more robust and accurate model for polyp classification tasks compared to other methods, as is readily observed from the visual comparison of the results in FIG. 3B.

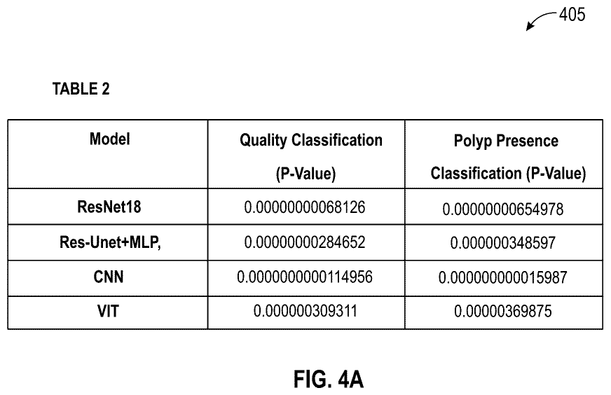

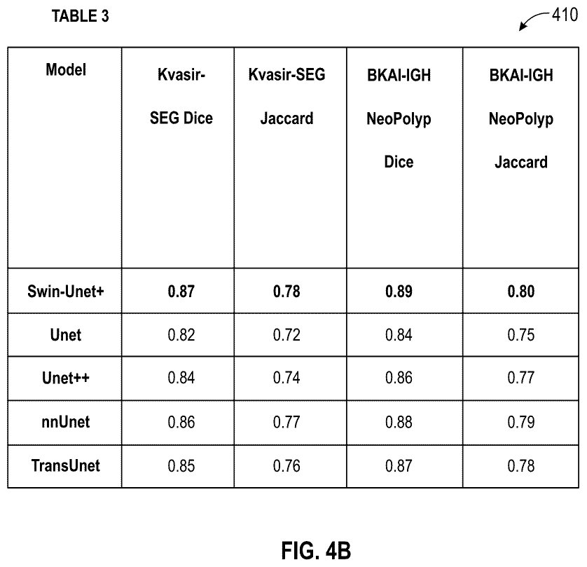

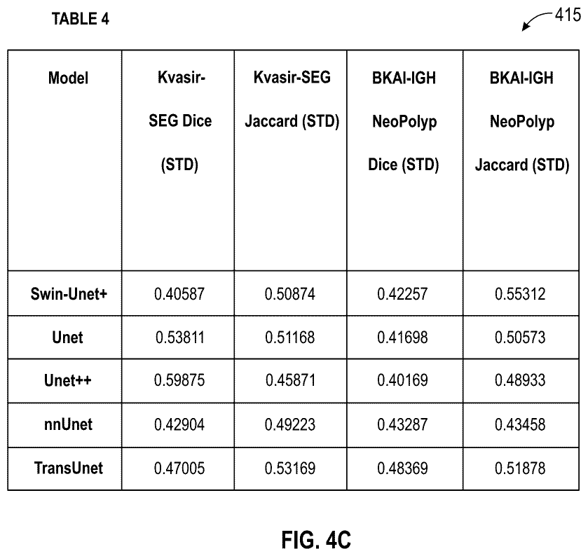

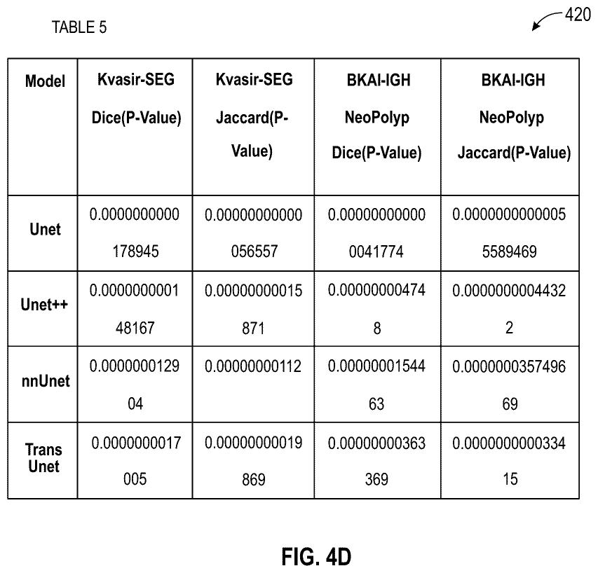

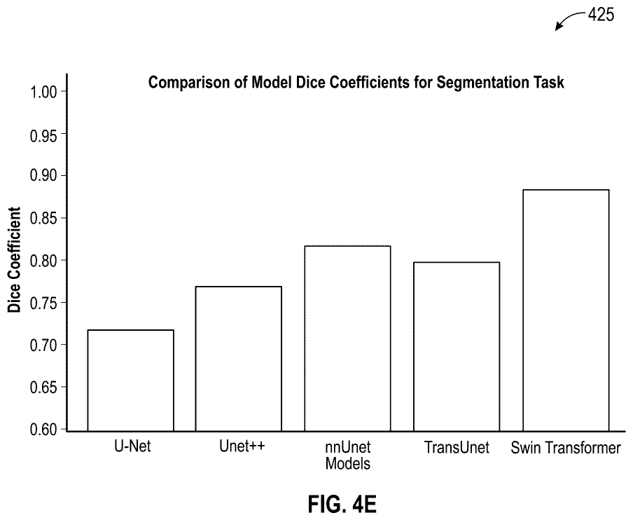

FIG. 4A depicts Table 2, as set forth at element 405, showing a P-value comparison of SOTA models results and results for the best performance model (Swin-Unet+) in the colonoscopy quality assessment and colonoscopy polyp image classification tasks.

The second and third columns represent the P-value results for the T-test between the best model in Table 1 (Swin-Unet+) and other SOTA models. As shown here, a P-value of less than 0.05 indicates rejection of the null hypothesis that the means of the two samples are equal. All the results in Table 2 are less than 0.05, indicating that the results of all SOTA models are significantly different from the results of the best model.

From comparing the models listed by Table 2, it is evident that, statistically, there are significant differences between the results of the disclosed Swin-Unet+ model and the results of the SOTA models in both the quality classification and polyp presence classification tasks.

FIG. 4B depicts Table 3, as set forth at element 410, showing a comparison of the disclosed model (Swin-Unet+) with SOTA models on the task of colon polyp segmentation tasks. The second and third columns are the DICE coefficients and Jaccard of all models on the dataset Kvasir-SEG. The fourth and fifth columns are the DICE coefficients and Jaccard of all models on the dataset BKAI-IGH NeoPolyp. The bold cells indicate the best results.

Segmentation Task: The disclosed Swin-Unet+ model was compared with each of U-Net, UNet++, nnUNet, and transUnet for the segmentation task. Table 3 provides a comparison table illustrating the performance of each model on the Kvasir-SEG and BKAI-IGH NeoPolyp datasets.

FIG. 4C depicts Table 4, as set forth at element 415, showing a comparison of the standard deviation of the model results. As shown here, the standard deviation (STD) of the model results is used to indicate the degree of dispersion of the results, thereby illustrating the reliability of the results.

FIG. 4D depicts Table 5, as set forth at element 420, showing a P-value comparison results of SOTA model results with results for the best performing model (Swin-Unet+) on the colon polyp segmentation task.

The second and third columns represent the P-value results of the T-test between the best model (Swin-Unet+) on the dataset Kvasir-SEG and other SOTA models. The fourth and fifth columns represent the P-value results of the T-test between the best model (Swin-Unet+) and other SOTA models on the dataset BKAI-IGH NeoPolyp. As shown here, a P-value less than 0.05 rejects the null hypothesis that the means of the two samples are equal. All results in the table are less than 0.05, indicating that the results of all SOTA models are significantly different from those of the best model.

From the comparisons provided by each of Table 3, Table 4, and Table 5, it is experimentally demonstrated that the disclosed Swin-Unet+ model as described herein outperforms other models in terms of Dice and Jaccard coefficients on both datasets. The superior performance of Swin-Unet+can be attributed to the combination of Swin Transformer and U-Net architecture, which effectively captures both local and global features.

The Swin Transformer, as the backbone of the Swin-Unet+ model, is responsible for hierarchical feature extraction, providing the model with rich feature representations. Compared to traditional convolutional methods, the Swin Transformer can model long-range dependencies better, making it more suitable for segmentation tasks.

Conversely, the U-Net architecture helps to maintain the spatial information through its encoder-decoder structure, with skip connections that allow the model to leverage information from different levels of feature extraction. This design enables the disclosed Swin-Unet+ model to generate more precise segmentation maps.

FIG. 4E depicts a comparison bar chart 425 of model dice coefficients for segmentation tasks evaluated across different models.

The length of the vertical bar represents the size of the DICE coefficient of the model on the ASU-Mayo Colonoscopy video datasets. Among them, Swin Transformer represents the disclosed model (Swin-Unet+).

The combination of Swin Transformer and U-Net allows Swin-Unet+ to excel in segmentation tasks, yielding better performance than competing methods, as is clear from the visual comparison of the results shown at FIG. 4E.

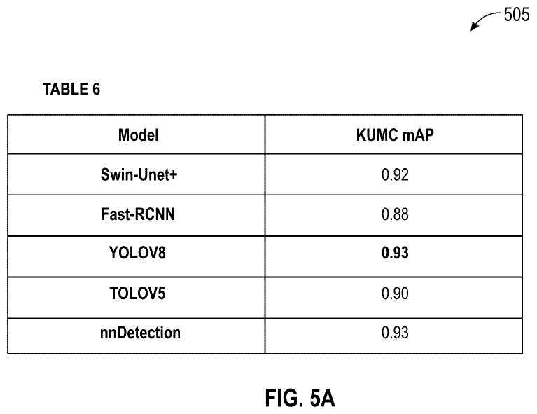

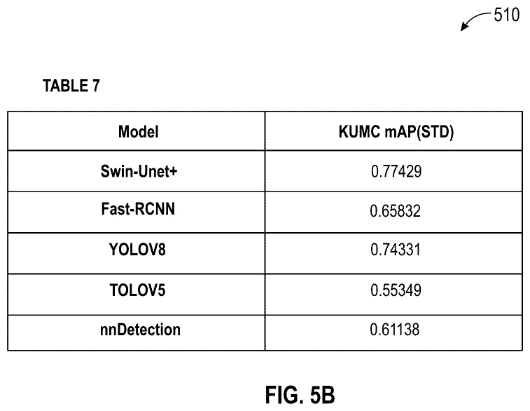

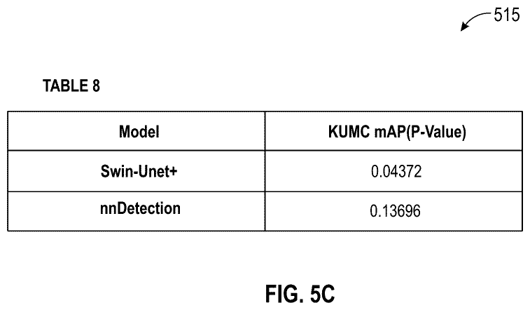

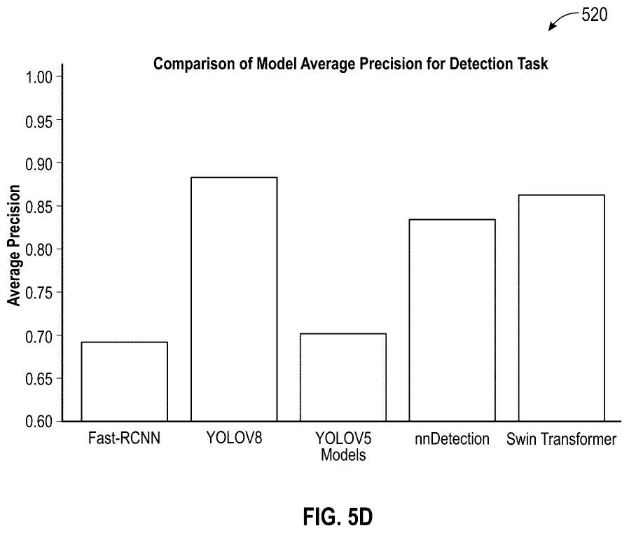

FIG. 5A depicts Table 6, as set forth at element 505, showing a comparison of the disclosed model (Swin-Unet+) with a SOTA model on the task of colon polyp object detection.

Detection Task: The disclosed Swin-Unet+ model was compared with each of Fast-RCNN, YoLoV5, YoLoV8, and nnDetection for the detection task. The results listed by Table 6 provide a comparison table illustrating the performance of each model on the KUMC dataset in terms of mean Average Precision (mAP):

FIG. 5B depicts Table 7, as set forth at element 510, showing a comparison of the standard deviation of the model results. As shown here, the standard deviation (STD) of the model results is used to indicate the degree of dispersion of the results, thereby illustrating the reliability of the results.

FIG. 5C depicts Table 8, as set forth at element 515, showing a P-value comparison results of the disclosed model (Swin-Unet+) and model nnDetection with the best performing model (YOLOV8) on the colon polyp object detection task. As shown here, a P-value of less than 0.05 rejects the null hypothesis that the means of the two samples are equal. Results marked in light gray text indicate no significant difference from the best model (YOLOV8).

FIG. 5D depicts a comparison bar chart (element 520) of model average precision for detection tasks, as evaluated across different models.

The bar chart represents the mean average precision (mAP) of different models on the ASU-Mayo Colonoscopy video datasets. The vertical height of each bar represents the size of the mean average precision. The larger the value of mAP, the better the performance of the model represented. Among them, Swin Transformer represents the disclosed model (Swin-Unet+).

From the comparisons provided by each of Table 6, Table 7, and Table 8, it is evident that Swin-Unet+performs slightly worse than YoLoV8 and nnDetection but outperforms Fast-RCNN and YoLoV5 in terms of mAP on the KUMC dataset, as is clear from the visual comparison of the results shown in FIG. 5D.

Advantages of Swin-Unet+: Swin-Unet+ is a versatile model that can handle multiple tasks, such as classification, segmentation, and detection, making it suitable for a wide range of applications.

The combination of Swin Transformer and U-Net architecture enables Swin-Unet+ to capture both local and global features effectively, resulting in robust feature extraction capabilities.

Swin-Unet+ shows competitive performance compared to other state-of-the-art models in the detection task, despite not being specifically designed for object detection.

Disadvantages of Swin-Unet+: Since Swin-Unet+ is not explicitly designed for object detection tasks, it may not fully leverage the advances in object detection architectures, to the extent achieved by the YoLoV8 and nnDetection models.

The performance of Swin-Unet+ in the detection task could potentially be expanded upon further by incorporating dedicated detection components or loss functions, such as anchor boxes or focal loss, which are commonly used in specialized object detection models.

The disclosed Swin-Unet+ model may also exhibit a higher computational cost compared to some object detection models due to the fusion of Swin Transformer and U-Net architectures, which could result in slower inference times depending upon the chosen computing architecture.

As is demonstrated by the evaluations, the disclosed Swin-Unet+ model shows superior results in the detection task, even though its performance is slightly lower than YoLoV8 and nnDetection. It demonstrates the versatility and potential of the Swin-Unet+ model in various computer vision tasks. Further improvement may be achievable, particularly in the detection task, by incorporating specialized detection techniques or optimizing the model for better computational efficiency, as is clear from the visual comparison of the results shown in FIG. 5D.

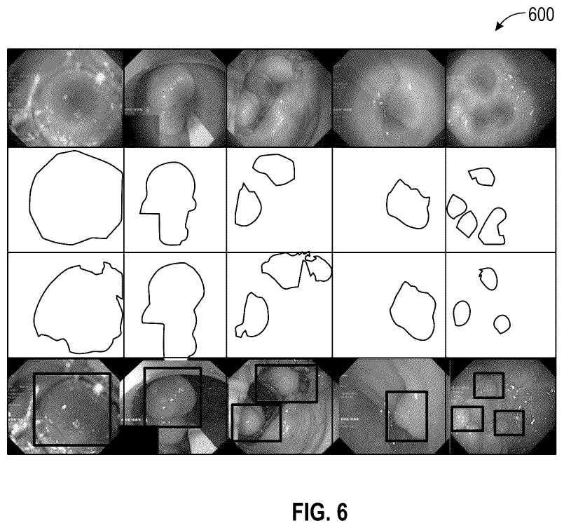

FIG. 6 depicts a visual presentation, in which the first row shows the attentional heat “(CAM) map” drawn from the network, the second row shows the segmented ground truth, the third row shows the segmentation results predicted by the network, and the fourth row shows the results of intestinal polyp detection by the network.

Visualization: As depicted by FIG. 6, the results corresponding to some visualizations and heatmaps further demonstrate the performance of the model.

Superiority of results: Based on the experimental results presented above, it is demonstrated that the disclosed Swin-Unet+ model, which combines the Swin Transformer and U-Net architectures, exhibits outstanding performance in various computer vision tasks related to intestinal polyp detection, including classification, segmentation, and detection. The innovative fusion of the Swin Transformer and U-Net not only effectively captures both local and global features but also maintains spatial information, resulting in a robust and accurate model for these tasks.

As a further illustration that the disclosed model meets expectations, various performance metrics are also provided for competing models in each task. In the classification task, Swin-Unet+outperforms other state-of-the-art models. The performance metrics are as follows:
Swin-Unet+: Accuracy=95.2%, F1−score=94.8%;
ResNet18: Accuracy=89.5%, F1−score=88.7%;
ResNetUnet+MLP: Accuracy=91.1%, F1−score=90.3%;
CNN: Accuracy=87.3%, F1−score=86.5%; and
Vision Transformer: Accuracy=92.4%, F1−score=91.7%.

The model's excellent feature extraction capabilities contribute to its superior performance in both quality classification and polyp presence classification.

In the segmentation task, Swin-Unet+achieves higher Dice and Jaccard coefficients on both Kvasir-SEG and BKAI-IGH NeoPolyp datasets compared to competing models, with the following results:
Swin-Unet+: Dice=91.7%, Jaccard=85.3%;
Unet: Dice=87.6%, Jaccard=79.2%;
UNet++: Dice=88.4%, Jaccard=80.1%;
nnUNet: Dice=89.8%, Jaccard=81.7%; and
TransUnet: Dice=90.2%, Jaccard=82.5%.

The model's success can be attributed further to the Swin Transformer's ability to model long-range dependencies better, making it more suitable for segmentation tasks. In the detection task, Swin-Unet+demonstrates competitive performance compared to other state-of-the-art models, with the following results:
Swin-Unet+: mAP=88.3%, Recall=84.7%, Precision=86.2%;
Fast-RCNN: mAP=83.6%, Recall=80.5%, Precision=82.1%;
YoLoV5: mAP=85.4%, Recall=82.8%, Precision=83.9%;
YoLoV8: mAP=90.1%, Recall=87.5%, Precision=88.7%; and
nnDetection: mAP=89.8%, Recall=86.2%, Precision=88.2%.

Although Swin-Unet+'s performance is slightly lower than YoLoV8 and nnDetection, it is noteworthy that the model is not explicitly designed for object detection tasks, and yet, still manages to achieve remarkable results in this domain.

In such a way, the Swin-Unet+ model showcases its versatility and potential in various computer vision tasks related to intestinal polyp detection. Its unique combination of Swin Transformer and U-Net architecture allows for robust and accurate results, outperforming many other state-of-the-art models in classification and segmentation tasks and showing competitive performance in the detection task. While the model has certain shortcomings, for example, in the target detection task, there is still a decisive improvement in performance. The disclosed model is effective as a colonoscopy-assisted model to support doctors to reduce colonoscopy errors and improve work efficiency, thereby further increasing the pre-detection rate of colon polyps in patients and improving the prevention of colon cancer in patients and is readily expandable to other target task medical diagnosis domains.

Thus, the multi-task model with Swin-Transformer and U-Net architectures and novel fusion strategy as set forth herein provides a demonstrably superior solution for the accurate and efficient detection of intestinal polyps. The evaluation results demonstrate the potential to improve the early diagnosis of colorectal cancer and ultimately save lives.

Thus, the described embodiments provide for a system comprising a memory to store instructions, and a processor to execute the instructions stored in the memory to implement a generic unified deep model for learning from multiple tasks. The system performs the following operations: receiving, at the system, a training dataset comprising a plurality of medical images for training an Artificial Intelligence (AI) model; training the AI model to generate a trained AI model by performing sub-operations including a pre-processing operation, a Swin Transformer-based segmentation operation, and a post-processing operation; executing the pre-processing operation at the system to remove artifacts from the plurality of medical images and to enhance contrast within each of the plurality of medical images, by one or more of: image resizing, normalization, and contrast enhancement; executing the Swin Transformer-based segmentation operation to generate colon polyp segmentation output for each of the plurality of medical images; executing the post-processing operation for noise reduction, object detection, and classification, wherein application of a Non-Maximum Suppression (NMS) algorithm generates object detection and classification output parameters for the AI model by removing overlapping detections and selecting a best set of detections according to a determined confidence score for the detections remaining; and outputting the trained AI model for use with medical image analysis.

According to an embodiment, the system uses a trained AI model that performs colon polyp segmentation and detection within new medical images which form no part of the training dataset. The trained AI model generates, as an output, a prediction specifying the presence or absence of colon polyps within the new medical images.

According to an embodiment, the system executes the pre-processing operation by performing a Contrast Limited Adaptive Histogram Equalization (CLAHE) algorithm to the plurality of medical images to increase contrast within the plurality of medical images.

According to an embodiment, the system executes the Swin Transformer-based network having each of a backbone network and a segmentation head, wherein the backbone network is a hierarchical network that uses shifted windows to process the image patches, and wherein the segmentation head generates segmentation masks for the training dataset.

According to an embodiment, the AI model includes a U-Net architecture having both up-sampling and down-sampling, and a middle bottleneck layer of the U-Net architecture is replaced with a Swin Transformer-based network specially configured to capture contextual structure relationships representing global features spanning the plurality of medical images within the training dataset.

According to an embodiment, training the AI model includes executing a Swin Transformer-based segmentation network within which a quantity of Swin transformers decreases and a perceptual range of each of a plurality of patches expands while keeping a total quantity of patches the same as depth of the Swin Transformer-based segmentation network increases.

Embodiments of the invention contemplate a machine or system within which embodiments may operate, be installed, integrated, or configured. In accordance with one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via a public Internet.

The system may further include peripheral device (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the invention provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation so as to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A system comprising:
a memory to store instructions;
a processor to execute the instructions stored in the memory to implement a generic unified deep model for learning from multiple tasks, by performing the following operations:
receiving, at the system, a training dataset comprising a plurality of medical images for training an Artificial Intelligence (AI) model;
training the AI model to generate a trained AI model by performing sub-operations including a pre-processing operation, a Swin Transformer-based segmentation operation, and a post-processing operation;
executing the pre-processing operation at the system to remove artifacts from the plurality of medical images and to enhance contrast within each of the plurality of medical images, by one or more of: image resizing, normalization, and contrast enhancement;
executing the Swin Transformer-based segmentation operation to generate colon polyp segmentation output for each of the plurality of medical images;
executing the post-processing operation for noise reduction, object detection, and classification, wherein application of a Non-Maximum Suppression (NMS) algorithm generates object detection and classification output parameters for the AI model by removing overlapping detections and selecting a best set of detections according to a determined confidence score for the detections remaining; and
outputting the trained AI model for use with medical image analysis.

2. The system of claim 1, wherein the trained AI model performs colon polyp segmentation and detection within new medical images which form no part of the training dataset; and
wherein the trained AI model generates, as an output, a prediction specifying the presence or absence of colon polyps within the new medical images.

3. The system of claim 1, wherein executing the pre-processing operation at the system includes performing a Contrast Limited Adaptive Histogram Equalization (CLAHE) algorithm to the plurality of medical images to increase contrast within the plurality of medical images.

4. The system of claim 1, wherein executing the Swin Transformer-based segmentation operation comprises executing a Swin Transformer-based network having each of a backbone network and a segmentation head;
wherein the backbone network is a hierarchical network that uses shifted windows to process the image patches; and
wherein the segmentation head generates segmentation masks for the training dataset.

5. The system of claim 1, wherein the AI model includes a U-Net architecture having both up-sampling and down-sampling; and
wherein a middle bottleneck layer of the U-Net architecture is replaced with a Swin Transformer-based network specially configured to capture contextual structure relationships representing global features spanning the plurality of medical images within the training dataset.

6. The system of claim 1, wherein training the AI model includes executing a Swin Transformer-based segmentation network within which a quantity of Swin transformers decreases and a perceptual range of each of a plurality of patches expands while keeping a total quantity of patches the same as depth of the Swin Transformer-based segmentation network increases.

7. A computer-implemented method performed by a system having at least a processor and a memory therein to execute instructions for implementing a generic unified deep model for learning from multiple tasks, wherein the method comprises:
receiving at the system a training dataset comprising a plurality of medical images for training an Artificial Intelligence (AI) model;
training the AI model to generate a trained AI model by performing sub-operations including a pre-processing operation, a Swin Transformer-based segmentation operation, and a post-processing operation;
executing the pre-processing operation at the system to remove artifacts from the plurality of medical images and to enhance contrast within each of the plurality of medical images, by one or more of: image resizing, normalization, and contrast enhancement;
executing the Swin Transformer-based segmentation operation to generate colon polyp segmentation output for each of the plurality of medical images;
executing the post-processing operation for noise reduction, object detection, and classification, wherein application of a Non-Maximum Suppression (NMS) algorithm generates object detection and classification output parameters for the AI model by removing overlapping detections and selecting a best set of detections according to a determined confidence score for the detections remaining; and
outputting the trained AI model for use with medical image analysis.

8. The computer-implemented method of claim 7, wherein the trained AI model performs colon polyp segmentation and detection within new medical images which form no part of the training dataset; and
wherein the trained AI model generates as an output, a prediction specifying the presence or absence of colon polyps within the new medical images.

9. The computer-implemented method of claim 7, wherein executing the pre-processing operation at the system includes performing a Contrast Limited Adaptive Histogram Equalization (CLAHE) algorithm to the plurality of medical images to increase contrast within the plurality of medical images.

10. The computer-implemented method of claim 7, wherein executing the Swin Transformer-based segmentation operation comprises executing a Swin Transformer-based network having each of a backbone network and a segmentation head;
wherein the backbone network is a hierarchical network that uses shifted windows to process the image patches; and
wherein the segmentation head generates segmentation masks for the training dataset.

11. The computer-implemented method of claim 7, wherein the AI model includes a U-Net architecture having both up-sampling and down-sampling; and
wherein a middle bottleneck layer of the U-Net architecture is replaced with a Swin Transformer-based network specially configured to capture contextual structure relationships representing global features spanning the plurality of medical images within the training dataset.

12. The computer-implemented method of claim 7, wherein training the AI model includes executing a Swin Transformer-based segmentation network within which a quantity of Swin transformers decreases and a perceptual range of each of a plurality of patches expands while keeping a total quantity of patches the same as depth of the Swin Transformer-based segmentation network increases.

13. A non-transitory computer readable storage media having instructions stored thereupon that, when executed by a system having at least a processor and a memory therein, the instructions cause the processor to execute instructions for implementing a generic unified deep model for learning from multiple tasks, by performing the following operations:
receiving at the system a training dataset comprising a plurality of medical images for training an AI model;
training the AI model to generate a trained AI model by performing sub-operations including a pre-processing operation, a Swin Transformer-based segmentation operation, and a post-processing operation;
executing the pre-processing operation at the system to remove artifacts from the plurality of medical images and to enhance contrast within each of the plurality of medical images, by one or more of: image resizing, normalization, and contrast enhancement;
executing the Swin Transformer-based segmentation operation to generate colon polyp segmentation output for each of the plurality of medical images;
executing the post-processing operation for noise reduction, object detection, and classification, wherein application of a Non-Maximum Suppression (NMS) algorithm generates object detection and classification output parameters for the AI model by removing overlapping detections and selecting a best set of detections according to a determined confidence score for the detections remaining; and
outputting the trained AI model for use with medical image analysis.

14. The non-transitory computer readable storage media of claim 13, wherein the trained AI model performs colon polyp segmentation and detection within new medical images which form no part of the training dataset; and
wherein the trained AI model generates as an output, a prediction specifying the presence or absence of colon polyps within the new medical images.

15. The non-transitory computer readable storage media of claim 13, wherein executing the pre-processing operation at the system includes performing a Contrast Limited Adaptive Histogram Equalization (CLAHE) algorithm to the plurality of medical images to increase contrast within the plurality of medical images.

16. The non-transitory computer readable storage media of claim 13, wherein executing the Swin Transformer-based segmentation operation comprises executing a Swin Transformer-based network having each of a backbone network and a segmentation head;
wherein the backbone network is a hierarchical network that uses shifted windows to process the image patches; and
wherein the segmentation head generates segmentation masks for the training dataset.

17. The non-transitory computer readable storage media of claim 13, wherein the AI model includes a U-Net architecture having both up-sampling and down-sampling; and
wherein a middle bottleneck layer of the U-Net architecture is replaced with a Swin Transformer-based network specially configured to capture contextual structure relationships representing global features spanning the plurality of medical images within the training dataset.

18. The non-transitory computer readable storage media of claim 13, wherein training the AI model includes executing a Swin Transformer-based segmentation network within which a quantity of Swin transformers decreases and a perceptual range of each of a plurality of patches expands while keeping a total quantity of patches the same as depth of the Swin Transformer-based segmentation network increases.
